(19) United States
(12) Patent Application Publication
(10) Pub. No.: US 2023/0363662 A1
(43) Pub. Date: Nov. 16, 2023

(54) TISSUE FLUID MEASUREMENT DEVICE

(71) Applicant: Mode Sensors AS, Trondheim (NO)

(72) Inventors: Terje SÆTHER, Trondheim (NO); Jørn KVÆRNESS, Oppdal (NO)

(73) Assignee: Mode Sensors AS, Trondheim (NO)

(21) Appl. No.: 18/029,178

(22) PCT Filed: Sep. 29, 2021
(86) PCT No.: PCT/EP2021/076809

§ 371 Date: Mar. 29, 2023

(30) Foreign Application Priority Data
Sep. 30, 2020 (NO) 20201071

(51) Int. Cl. A61B 5/0537; A61B 5/00

(52) U.S. Cl. CPC A61B 5/0537; A61B 5/6833

(57) ABSTRACT

A method for determining a change in cell volume with a sensor device having four electrodes. The method comprises the following steps: ⋅applying a current with a known cycle of different known frequencies in time series on an outer pair of electrodes; measuring a resulting variating potential over an inner pair of electrodes; ⋅calculating a total impedance absolute value from said measured potential; ⋅storing each total impedance against frequency value in a memory; ⋅reading a frequency value belonging to at least one impedance value of a subsequent cycle; ⋅reading a frequency value belonging to the same at least one impedance value of a previous cycle; ⋅comparing said frequency values and determining a difference between the frequency values; and ⋅providing a time series of differences in frequency across said time series, an increasing frequency difference across said time series indicating a relative increase or decrease of cell volume.

### TECHNICAL FIELD

The present invention relates to a method and a device for measuring tissue fluid, especially non-invasive measurement of human tissue impedance. The device is particularly suitable for determining hydration level of a person, but it may also be used to observe disease processes included in the Renin-agiotensin-aldosterone system such as: inflammatory processes (obesity, rheumatic disorders, osteoarthritis, cancer, stroke and heart attack, rhabdomyolysis), hypertension and atheriosclerosis, diabetes and pancreatic disease, liver disease and pancreas. The apparatus can be used as a tissue fluid monitoring device, a body fluid measurement device or a device for monitoring fluid volume and tonicity.

### BACKGROUND ART

A huge range of body impedance measurement devices are known in the art, ranging from devices to measure full body impedance to determine muscle/fat ratio to measurement of skin impedance in order to determine hydration level.

There are three principle ways of measuring resistance and reactance measurements (often referred to as bioimpedance measurements):
single frequency bioimpedance (SFBI),
bioimpedance spectroscopy (BIS) which uses a number of discrete frequencies identical to multi frequency bioimpedance (MFBI), and
bioimpedance vector analysis (BIVA).

All these techniques base their measurements on Cole-Cole or Nyquist plots, then transform the measurements through algorithms into total body volume of water (TBW) and extracellular volume (ECV), and finally use these to calculate intracellular volume (ICV). Whole body bioimpedance measurements can then provide a body analysis beyond hydration. Series of publications and review articles all have shown that bioimpedance measurements processed as described above can both overestimate and underestimate hydration with significant margins of error. Furthermore, different diseases and disease states results in even larger errors and thus very low specificity.

Only a few products utilize the time-resolved changes to improve their calculation, and none of the prior art devices consider changes in cell size. Since ICV is a subtraction of ECW (Extra Cellular Water) from TBW, and TBW calculation is very error prone, the ICV calculation is equally hampered by a number of confounding factors.

Some examples of prior art references are:

WO02016030869A1, which describes a four-probe impedance skin measurements over various frequencies. The reference claims that intra and extra cellular contributions are decorrelated by the measurements.

US20170172484A1, which also related to skin measurements using several frequencies.

US20180168459A1, which related to skin measurements using one or more frequencies between 100 Hz and 1 MHz. The reference claims that a distinction can be made between intra and extra cellular water contents.

WO2016005915A3, which also describes skin measurements, using at least two frequencies. This reference also claims that a precise distinction can be made between extra and intra cellular water. The loss of hydration can be determined by a relationship between these two.

U.S. Pat. No. 10,610,111, which also relates to skin measurements using a wide range of frequencies.

U.S. Pat. No. 10,531,842, which also describes determination of intra and extra cellular water. In the method of this reference the well-known Cole-Cole model is used.

The “ESPEN guideline on clinical nutrition and hydration in geriatrics” of 2018 (available from Elsevier.com or https://www.clinicalnutritionjournal.com/article/S0261-5614(18)30210-3/fulltext) lists 82 recommendations for clinical nutrition and hydration in older persons to prevent malnutrition and dehydration. Recommendation 64 is that all older persons should be screened for low-intake dehydration when they contact the health care system. Raised serum osmolality (higher concentration of chemicals such as sodium, chloride, bicarbonate, proteins, and glucose due to dehydration) may double the risk of mortality.

Another recommendation is to directly measure serum or plasma osmolality to identify dehydration. This is, however, a time-consuming procedure that can be done only with certain time intervals. It cannot detect rapid changes in hydration level.

In recommendation 69, the guidelines, however, discourages the use of bioelectrical impedance to assess hydration status, as this “has not been shown to be usefully diagnostic”. The guidelines refer in this respect to a Cochrane review, “Clinical symptoms, signs and tests for identification of impending and current water-loss dehydration in older people”, of 2015 (available from Cochrane Library—https://www.cochranelibrary.com/cdsr/doi/10.1002/14651858.CD009647.pub2/full). The Cochrane review analyzed 67 tests, including bioelectrical impedance measurements. The review found no evidence of the utility of bioelectric impedance in assessment of hydration in older persons.

Although the above prior art examples of bioelectric impedance measurement methods all claim to be able to determine dehydration, or hyperhydration, with accuracy, the ESPEN guideline and the Cochrane review show that it is not that simple. The signal to noise level is very high for these measurements, especially in the high frequency range, around 500 kHz or above. This means that, in practical use, none of the methods described above are able to determine the ratio between intra and extra cellular water with a high enough accuracy to be used to safely monitor patient hydration level.

Conventional noise reduction requires sophisticated and bulky electronics and large computational power. This means that the sensor device attached to the skin must be coupled to an external module. Hence, the total arrangement will be bulky. This means that the patient needs to be largely immobile while being monitored. It also means that the device will be expensive and has to be moved between patients to defend the costs.

### SUMMARY OF INVENTION

The present invention therefore has as its main objective to increase the accuracy of the hydration level determination sufficiently for the method to be useful in monitoring of hydration level of patients.

It is also an objective to provide for a sensor device that can fit in a patch to be attached to the skin of a patient without the need of being constantly coupled to an external device, and which can be single use.

This is achieved by a method for determining a change in cell volume with a sensor device having four electrodes; applying a current with a known cycle of different known frequencies in time series on an outer pair of said electrodes; measuring a resulting variating potential over an inner pair of said electrodes; wherein said method further comprising: calculating a total impedance absolute value according to per se known methods from said measured potential at each frequency in said time series; storing each total impedance against frequency value for said time series of cycles in a memory, such as a random access memory or non-volatile memory; reading a frequency value belonging to at least one impedance value of a subsequent cycle; reading a frequency value belonging to the same at least one impedance value of a previous cycle; comparing said frequency values and determining a difference between the frequency values; and providing a time series of differences in frequency across said time series, an increasing frequency difference across said time series indicating a relative increase or decrease of cell volume.

A better estimate can be achieved by reading respective frequencies belonging to a plurality of impedance values of a previous and subsequent cycle, determining differences between each frequency value for the same impedance value, and calculating an average difference between frequency values of said previous and subsequent cycles.

Extra cellular bio impedance and intra cellular bio impedance can then be determined from a Cole-Cole plot.

By curve-fitting the measured variating potential over the cycle of frequencies to an empirical data model through a per se known numerical method of curve-fitting, such as the least mean squares technique, where parameters to be used to determine a change of cell volume are extracted from said empirical model, said empirical model being the following approximation:

$$\vert Z ( f ) \vert \approx \sqrt[γ]{\frac{{R}_{E}^{γ} + {( {R}_{T} \cdot α \cdot f )}^{γ}}{1 + {( α \cdot f )}^{γ}}}$$

where |Z(f)| is the absolute value (magnitude) of the impedance, γ is a slope parameter derived from the steepness of a magnitude plot determined as an empirical value from a large set of measurements, R_{E} is extra cellular resistance, R_{I} is intracellular resistance, R_{T} is the total resistance, f is the frequency, and α is a frequency dependent factor of the cell volume, and determining a change in cell volume, the change in cell volume being according to the following relationship:

$$Cell\_volume \sim \frac{α}{{R}_{E} + {R}_{I}}$$

a sufficiently accurate estimate of cell volume can be achieved without the need for considerable computational power.

To eliminate or reduce the impact of measurement errors the measured potential over the frequency cycle is compared with an upper boundary curve and a lower boundary curve, where the boundary curves are determined such that the measured impedance on a higher frequency is allowed to be within A%, typically 90%, to B%, typically 101% ,of the closest lower frequency and that a series of potential measurements over the cycle of frequencies is rejected if any of the measurements fall outside on or both said boundary curves.

An empirical data model can be achieved or improved by using precise reference measurement with a high-quality instrument.

The slope parameter can also conveniently be determined by use of said precise reference measurements.

Noise can be reduced by passing a true signal from a signal and noise mix resulting from the measured potential through a Fourier transform for each of said known frequencies, using the following formula:

ŝ= [symbol] (s+n)

where ŝ is an estimate of the signal without noise, s is the pure signal and n is the noise.

The features of the present invention enables implementation of the electrodes and a processor capable of calculating an estimate of cell volume change using the empirical data model into a patch to be adhered to the skin of a patient and still achieve sufficient accuracy of the results.

The results are used to determine underhydration and overhydration by comparing the calculated estimate of cell volume change and total impedance absolute value change to threshold values and when any of the cell volume change and/or the total impedance value change exceeds said threshold values, send an alert indicating underhydration or overhydration to a receiver unit. Thereby a simple and convenient instrument for monitoring of a patient is achieved.

As a further improvement a change in either extra cellular bio impedance or intra cellular bio impedance can be monitored. Thereby an even more precise monitoring of the patient can be achieved.

### BRIEF DESCRIPTION OF DRAWINGS

The present invention will now be explained in connection with exemplary embodiments illustrated by the accompanying drawings, in which:

FIG. 1 illustrates schematically the principles of the sensor of the present invention,

FIG. 2 shows a flowchart illustrating the method of the present invention according to a preferred embodiment,

FIG. 3 shows an example of a signal that has been run through Fourier transform to estimate the true signal and hence also the noise,

FIG. 4 shows the impedance curve as a function of frequency, as well as the set boundaries for the curve,

FIG. 5 shows a circuit diagram illustrating the bioimpedance of a single cell,

FIG. 6 shows a Cole-Cole plot of reactance versus resistance for a single cell,

FIG. 7 is a plot of the absolute value of the bioimpedance of a single cell versus frequency,

FIG. 8 illustrates different impedance versus frequency plots, similar to the single curve in FIG. 7, with different slope parameters,

FIG. 9 shows a plot of three different frequency cycles of bioimpedance versus frequency where the cell volume has changed between the cycles,

FIG. 10 shows the curves of FIG. 9 after the end points having been merged to eliminate shifts along the Y-axis,

FIG. 11 shows a plot similar to FIG. 8 but with two different types of tissue,

FIG. 12 shows the results of an experiment on a patient, where cell volume is plotted against time, and

FIG. 13 shows the results of a second experiment on a patient.

### DETAILED DESCRIPTION OF THE INVENTION

The method of the present invention applies a 4-electrode sensor device. 4-electrode sensors are considered the most accurate and versatile devices for body impedance measurements.

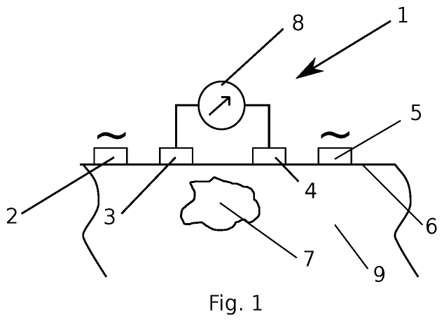

Although the function of such 4-electrode devices is well-known in the art (it was invented already in 1861 by Lord Kelvin), it will be briefly explained, referring to FIG. 1, which illustrates the principle. The sensor device 1 comprises four electrodes 2, 3, 4, 5, which are attached to a base and adapted to be placed in close contact with the skin 6 of the patient.

A constant current I(f) with a known frequency f is applied on the outer electrodes 2, 5, as illustrated by the tildes above the electrodes in FIG. 1. This will give a potential difference between the outer electrodes 2, 5 and between the inner electrodes 3, 4. Both potential differences are used by the patch. The potential difference between the outer electrodes is used to determine if the patch has good connection with the skin. The inner electrodes 3, 4 potential is measured by voltmeter 8, preferably with a high impedance variable voltage gain amplifier. As it is high impedance no current will flow through the inner electrodes and thus the potential difference, U(f), that is measured is due to the impedance in the tissue 9 and cells 7. The bio impedance is given as:

$$Z ( f ) = \frac{U ( f )}{I ( f )}$$

where Z(f) is the impedance, U(f) is the measured voltage between the two inner electrodes 3, 4 and I(f) is the current applied to the two outer electrode 2, 5.

So far, the method is conventional and theoretically very simple.

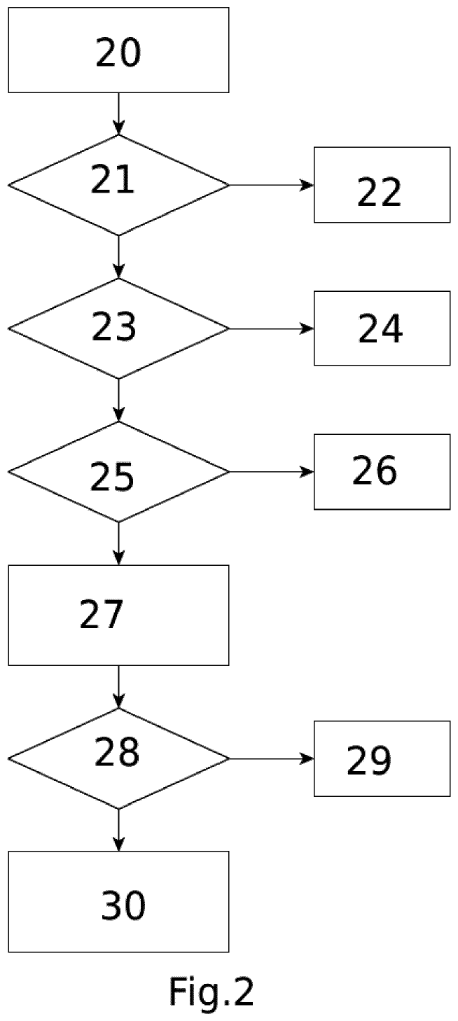

To avoid false measurements several quality checks of the signal is put into action, the flowchart for the measurement system is given in FIG. 2.

In the first step 20, the bio impedance Z(f) is measured according to the conventional method described above. A sinewave current with a known frequency and a duration of a few milliseconds is applied to the outer electrodes 2, 5. The resulting voltage signal measured by the voltmeter 8, amplified by a variable gain amplifier and digitized by an analog to digital converter (not shown). Subsequently, the results are analyzed by an embedded microcontroller (not shown). About 20 different frequencies may be applied to make up a complete measurement cycle. These measurement cycles are repeated and the interval between the cycles are typically between a few seconds to minutes and may be adaptive. The frequencies used for measurement are typically but not limited to 1 kHz to 500 kHz.

The sensor device is preferably embedded in a patch (not shown) that can be attached by an adhesive to a suitable location on the patient skin, such as between the shoulder blades. To ensure that the electrodes have good connection with the skin of the patient, the voltage potential across the two outer electrodes 2, 5 is measured at one or more frequencies. This potential is used to check for good skin connection (as illustrated by item 21).

If the potential difference between the two outer electrodes 2, 5 is outside a predetermined threshold the measurement cycle is discarded, as shown by item 22.

If several cycles in sequence are discarded, an alarm may be issued for a caretaker to check the patch.

Accepted signals are digitized and as the frequency of the signal is known, an estimate of the signal can be found by using Fourier transform. The measured signal, s+n (s=signal, n=noise), has both the wanted signal s, and noise n, which comes from the body itself and the measurement system. An estimate of the signal is found by Fourier transform.

As is well-known in the art, Fourier transform done by the following formula:

ŝ= [symbol] (s+n)

where ŝ is an estimate of the signal without noise, s is the pure signal and n is the noise.

Since Fourier transform is a filtering function the estimated signal will always be smaller than the original signal.

ŝ≤s+n

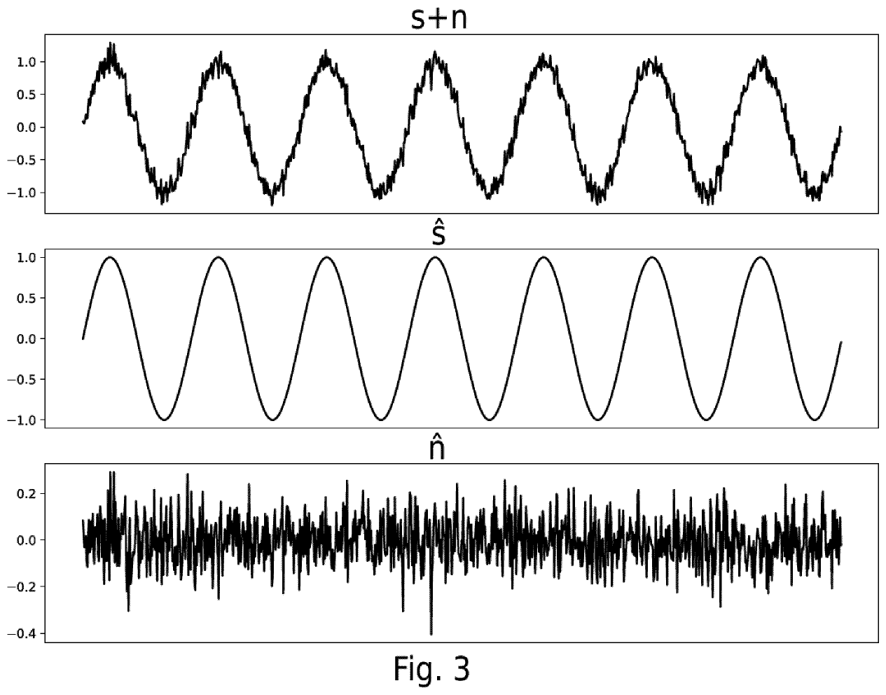

FIG. 3 shows an example of a Fourier transformation. The upper graph shows a measured signal, which contains a mixture of the desired signal and noise. As can be seen, the measured signal is a rough sinus curve with irregular ripples transposed thereon. The middle graph shows the estimated signal after a Fourier transformation. In this case the estimated signal is a clean and even sinus curve. The lower graph shows the estimated noise, which is the remainder of the measured signal after the estimated signal has been removed, i.e. the noise can be estimated by the following equation:

\hat{n}=s+n−ŝ

where

\hat{n}≥0

If the noise level is above a certain threshold the measurement cycle is rejected (see items 23 and 24 in FIG. 2). This is done even if the signal to noise ratio may be good, because there may be strong noise close to the measurement frequency and this noise will cause false interpretation of the signal estimate. If the noise is low the estimated signal is close to the real signal and it is possible to set

ŝ≈s

The threshold for setting the acceptable noise level may be fixed, adaptive or both. An adaptive threshold may be achieved by using the best measurements to determine a noise floor. The adaptive threshold may be a certain level above this floor, such 3-12 dB higher.

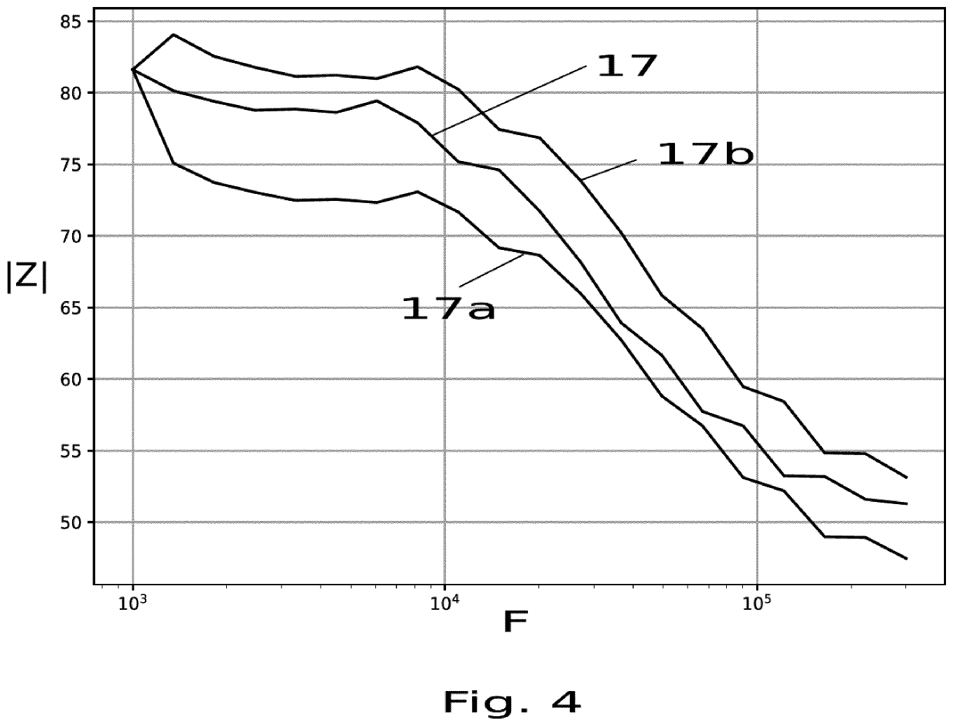

The next step in the analysis is assessing the curve shape of measured impedance (i.e. estimated signal) v. frequency (frequencies applied to the outer electrodes 2, 5). An exemplary curve is shown in FIG. 4. The impedance will always fall with increasing frequency. The measured curve (after Fourier transformation) is shown as the middle curve 17. As shown, the impedance as a function of frequency has a distinct curve form where the impedance falls with increasing frequency. The method of the invention therefore includes a step 25 applying a function that creates dynamic boundaries that the curve must stay within. These boundaries are shown as the lower curve 17a and the upper curve 17b. If several measurements making up the curve 17 are outside these boundaries, the measurement cycle is rejected (see items 25, 26 in FIG. 2).

The lower boundary 17a can conveniently be determined by allowing the impedance to fall a maximum value from one frequency to the next higher frequency, such as 5-20% of the previous lower frequency.

The upper boundary 17b can be determined by allowing the impedance only to increase slightly from the previous lower to the next higher frequency, such as 1-2% increase between the lower and the higher frequency.

The boundaries 17a, 17b are determined based on experience data and may be updated as new data sets are retrieved. The same applies for the threshold of how much of the curve 17 that may lie outside the boundaries before it is rejected.

In the next step (see item 27 in FIG. 2) of the method of the invention, the measured curves Z(f) are curve-fitted to the proprietary data model used in the present invention through a least mean squares (LMS) model. If the error is above a threshold the measurement cycle is discarded as illustrated by items 28 and 29 in FIG. 2. The data-model is created by using precise reference measurements with high quality instruments, such as Zurich Instruments MFIA 50 okHz/5 MHz Impedance Analyzer or ScioSpec ISX3 Electrical Impedance Spectroscopy.

In the final step (see item 30 in FIG. 2), four values are extracted from every measurement cycle; the extra cellular bio impedance, the intra cellular bio impedance, a parameter used for calculation of cell volume and the slope parameter.

Now the method of obtaining the cell volume will be explained.

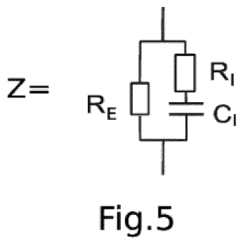

FIG. 5 shows a textbook representation of the bio impedance Z of a single cell. R_{E} represents the extra cellular resistance, R_{I} represents the intracellular resistance and C_{I} represents the cell membrane capacitance.

By solving the following equation:

$$Z ( w ) = \frac{{R}_{E} ( {C}_{I} {R}_{I} iw + 1 )}{{C}_{I} {R}_{E} iw + {C}_{I} {R}_{I} iw + 1}$$

where Z(w) is the total impedance of a single cell as a function of angular frequency and iw is the imaginary angular frequency, and then splitting the equation in resistance and reactance and introduce some variable substitution to simplify equation, the result is:

$$Z ( f ) = \frac{{R}_{E} + {R}_{T} {α}_{0}^{2} {f}^{2}}{{α}_{0}^{2} {f}^{2} + 1} - i \frac{{α}_{0} f ( {R}_{E} - {R}_{T} )}{{α}_{0}^{2} {f}^{2} + 1}$$

where α₀ is a frequency dependent factor of the single cell volume, f is the frequency, R_{T} is the total resistance, defined as:

$${R}_{T} = \frac{{R}_{E} \cdot {R}_{I}}{{R}_{E} + {R}_{I}}$$

and the frequency dependent variable α₀ is:

$${α}_{0} = 2 π {C}_{I} \frac{{R}_{E} {R}_{I}}{{R}_{T}}$$

### and Z(f)=R+iX

where R is Resistance and X is Reactance.

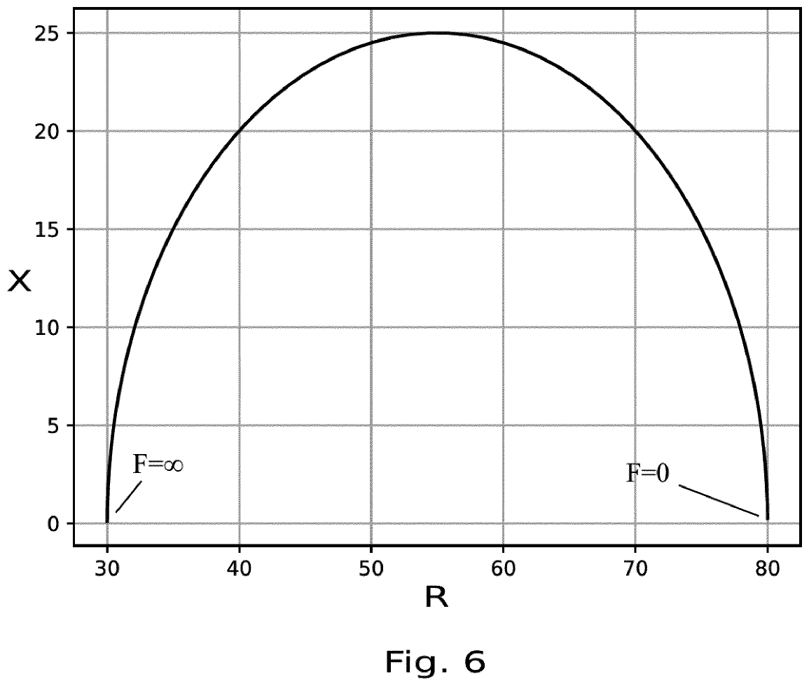

This is what prior art bioimpedance instruments measure, and the result may be shown as a Cole-Cole plot (aka Nyquist plot), as the example shown in FIG. 6, where the Resistance and Reactance are shown in the same diagram.

Important properties of this diagram are that at zero (i.e. below approximately 1 kHz) frequency the extracellular resistance can be read out of the diagram as R_{E}=Z(0). and at infinite frequency (i.e. above approximately 1 MHz) the total resistance can be read out as R_{T}=Z(∞).

The sensor device to be used for performing the present invention cannot measure phase but it can measure the magnitude, which is given as:

|Z(f)|=\sqrt{Resistance²+Reactance²}

which when the parameters are inserted, reads as follows:

$$\vert Z ( f ) \vert = \frac{\sqrt{{R}_{E}^{2} + {R}_{T}^{2} {α}_{0}^{2} {f}^{2}}}{\sqrt{{α}_{0}^{2} {f}^{2} + 1}}$$

where |Z(f)| is the absolute value (magnitude) of the impedance.

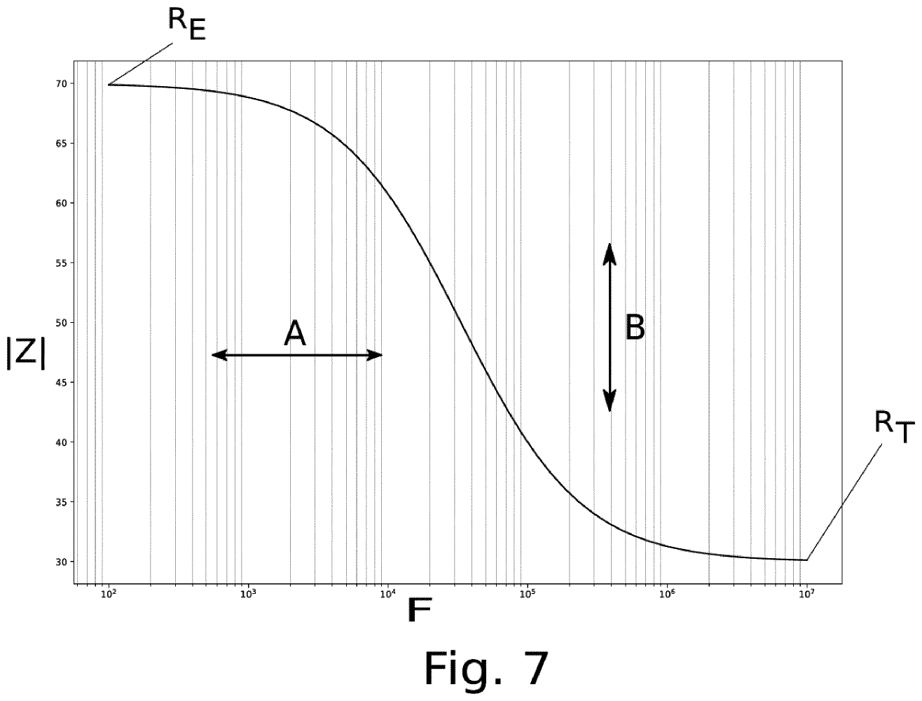

Plotting the magnitude of the above equation results in the graph of FIG. 7.

To be able to extract the three unknowns of the above equation, a, R_{E}, R_{T}, the minimum requirement is to measure the impedance at three different frequencies. But this is true only for a single cell. Also, if these three variables are extracted, the resistance and reactance can be reconstructed.

Since there are a huge number of cells within the measured part of the skin, with different sizes, the resulting equations will be very complex, if all aspects are to be taken into account.

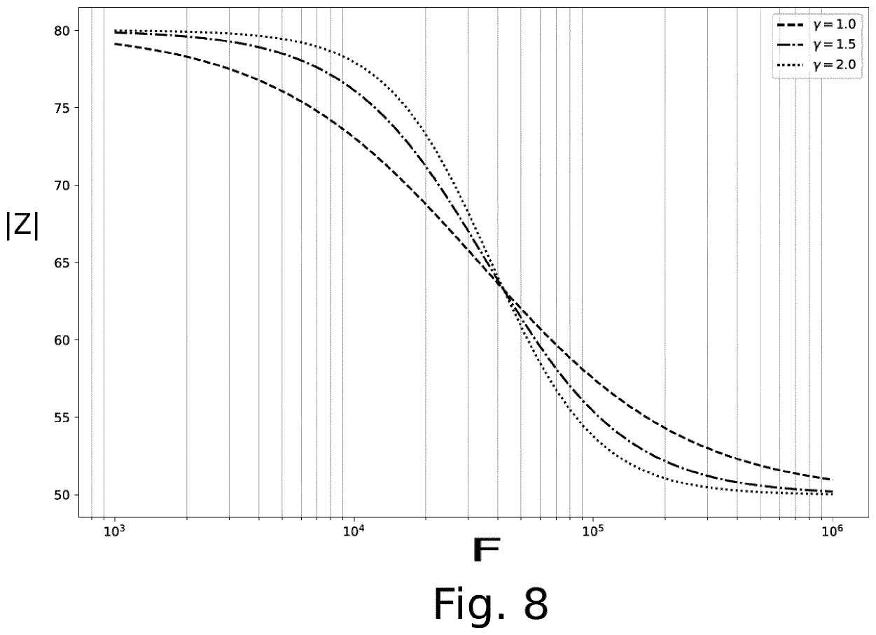

It is however an object of the present invention to implement the method into a device that is battery operated and has limited computational power. Thus a simple and good approximation needs to be developed. It has been found that the following equation will give a good approximation:

$$\vert Z ( f ) \vert \approx \sqrt[\begin{matrix}γ \\ \end{matrix}]{\frac{{R}_{E}^{γ} + {( {R}_{T} \cdot α \cdot f )}^{γ}}{1 + {( α \cdot f )}^{γ}}}$$

where γ is the slope parameter, i.e. the steepness of the magnitude plot. The value is an empirical value chosen from a large set of measurements. Measurements are done at the same place on the body on a large number of different patients. The location of musculus trapezius has been found to be a good place to make measurements, as this will provide consistent results. An example of plots with different slope parameters is shown in FIG. 8, where the steepest plot has a slope parameter of 2,0 and the least steep plot has a slope parameter of 1,0.

If the graph of FIG. 7 shifts along the X axis (see double arrow A) this indicates a change in cell volume, i.e. a shift to the left means that a gets larger and indicates a larger cell volume. A shift to the right indicates that a gets smaller and the cell volume is reduced. When there is a change in cell volume it is likely that the bio-impedances R_{E} and R_{T} change as well, which is seen by a shift along the Y axis, as indicated by the double arrow B. Thus, the correct cell volume will not be estimated from α alone but a combination of the three extracted parameters:

Cell_volume=function_of(α,R_{E},R_{T})

One example of this may be expressed as follows:

$$Cell\_volume \sim \frac{α}{{R}_{E} + {R}_{I}}$$

The valitidy of the model was tested. 93 sensors on a group of volunteers and the adjusted “Coefficient_of_determination”, (R squared) was used to validate the mode as illustrated in FIG. 8a. The adjusted R squared (R2) is used as it take into account the number of fitting parameters. Ref: https://en.wikipedia.org/wiki/Coefficient of determination

A latter version of the sensor device is capable of measure resistance, reactance and phase information thus a model can be constructed based on the impedance model.

A complex model may be found based on the following:
From paragraph [0058]

$$Z ( f ) = \frac{{R}_{E} + {R}_{T} {α}_{0}^{2} {f}^{2}}{{α}_{0}^{2} {f}^{2} + 1} - i \frac{{α}_{0} f ( {R}_{E} - {R}_{T} )}{{α}_{0}^{2} {f}^{2} + 1}$$

From paragraph [0060]

Z(f)=R+iX

This gives the general behaviour of the ideal single cell model, in particular it shows the expresiions of the resistance and reactance separated.
From paragraph [0070], with many cells the model is given as

$$\vert Z ( f ) \vert \approx \sqrt[\begin{matrix}γ \\ \end{matrix}]{\frac{{R}_{E}^{γ} + {( {R}_{T} \cdot α \cdot f )}^{γ}}{1 + {( α \cdot f )}^{γ}}}$$

For sake of principle and simplification of the equations set:

$$γ = 1$$

$$\vert Z ( f ) \vert \approx \frac{{R}_{E} + ( {R}_{T} \cdot α \cdot f )}{1 + ( α \cdot f )}$$

Introducing resistance and reactance

$${\vert Z ( f ) \vert}^{2} = {R}^{2} + {X}^{2} = \frac{{R}_{E}^{2} + φ {R}_{E} {R}_{T} α f + {{R}_{T}^{2} ( α f )}^{2}}{{( 1 + α f )}^{2}} + \frac{( 2 - φ ) {R}_{E} {R}_{T} α f}{{( 1 + α f )}^{2}}$$

And separate the resistance and reactance

$$R = \frac{\sqrt{{R}_{E}^{2} + φ {R}_{E} {R}_{T} α f + {{R}_{T}^{2} ( α f )}^{2}}}{( 1 + α f )}$$

$$X = \frac{\sqrt{( 2 - φ ) {R}_{E} {R}_{T} α f}}{( 1 + α f )}$$

$$Phase = {tan}^{- 1} ( \frac{X}{R} )$$

Hence by introducing another variable, φ, a complex model with resistance, reactance and phase is developed. The variable φ is found by curve fitting the resistance against the measured resistance values as illustrated in FIG. 8b, where Imaginary impedance corresponds to the reactance.

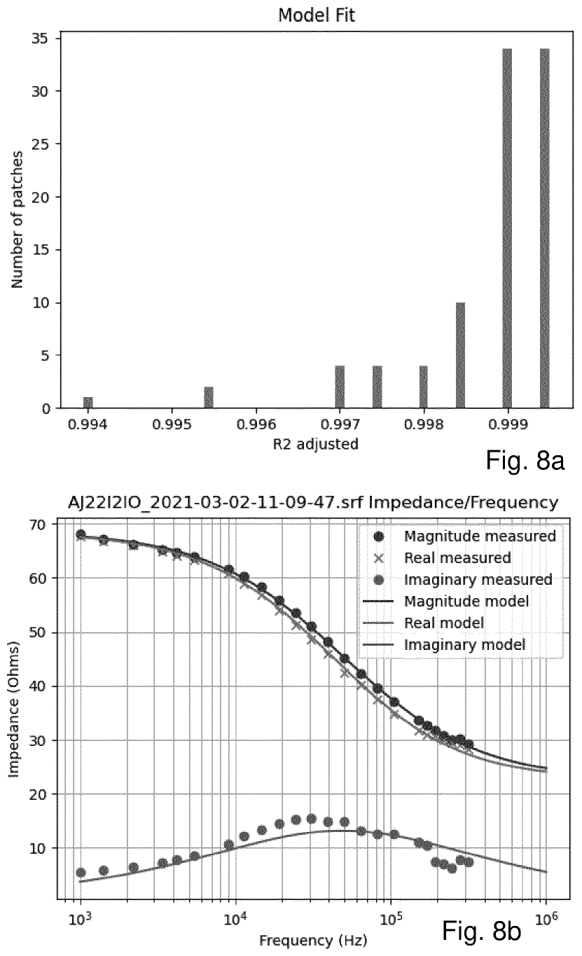

FIG. 9 illustrates a plot of three different frequency cycles of bioimpedance versus frequency. As is evident, the curve shifts both along the X and Y axis between the frequency cycles. As explained in connection with FIG. 7, this is an indication of a change in both cell volume and impedance between the cycles.

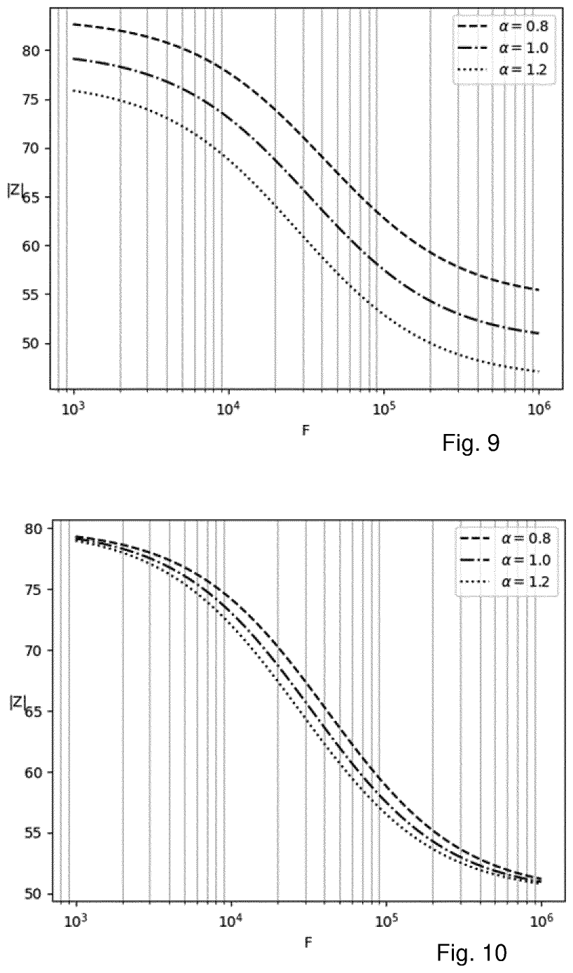

In FIG. 10 the end points of the three curves in FIG. 9 have been merged, i.e. the change in bioimpedance has been suppressed. By doing this the shift of the curve along the X axis becomes clearer, and it is easier to determine that a change in cell volume has occurred.

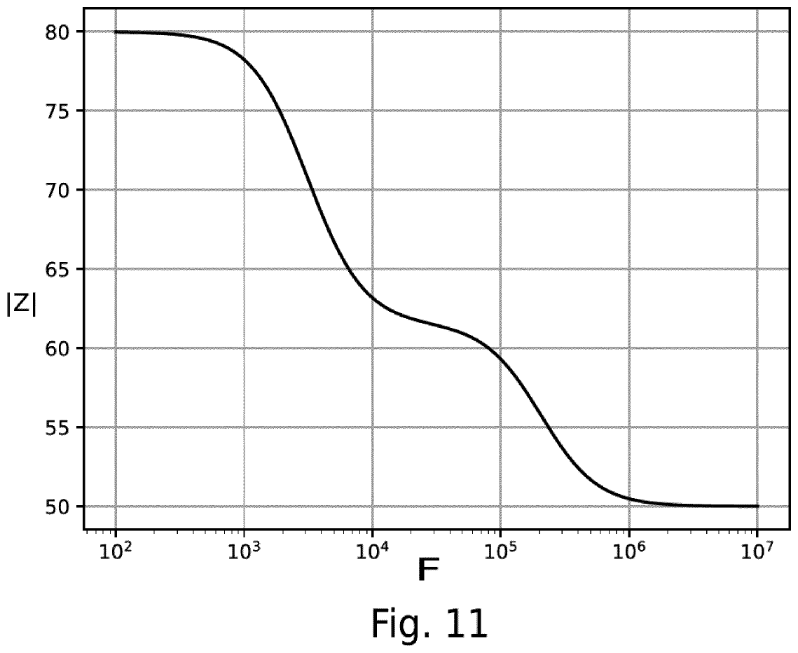

If there are two distinct cell types the curve form may have the shape shown in FIG. 11.

If there are more than one cell type, the resulting value is just the sum of the admittances for each cell type.

A simplified expression for this condition can by summing the admittances:

$${Z ( f )}_{total} = \frac{1}{\frac{1}{{Z ( f )}_{tissue 1}} + \frac{1}{{Z ( f )}_{tissue 2}}}$$

where tissue1 and tissue2 are two different types of cells.

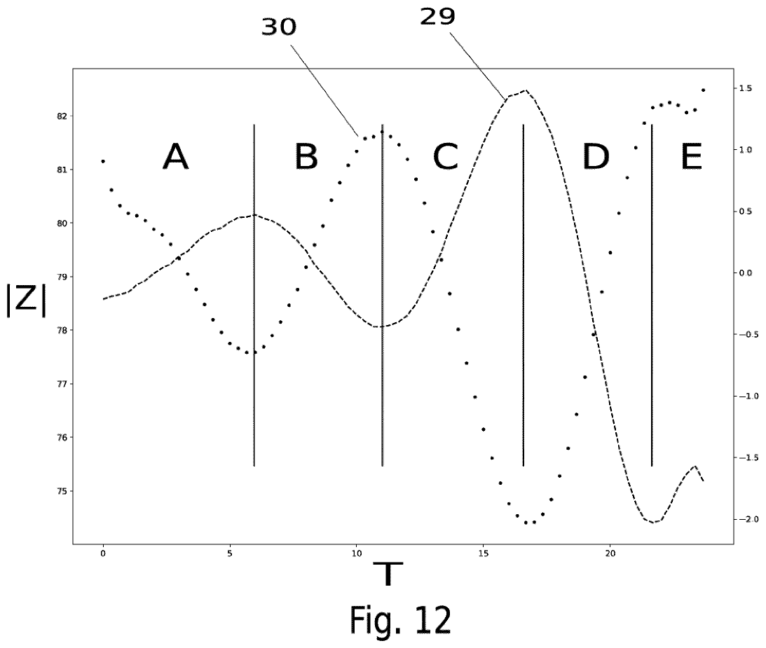

A real example of measurements is shown in FIG. 12.

FIG. 12 shows an idealized example for illustration purposes. It is based on actual measurements but simplified for ease of understanding.

Bio impedance is an absolute number, the cell size is normalized with the standard score method, which is a well-known method per se in statistical analysis.

In FIG. 12 the patient goes through five different phases A-E, signified by different behaviour of the patient, as will be described below. In the figure, extra cellular impedance is shown by the graph 29 and cell size is shown by the graph 30.
A—The patient is sleeping. The impedance 29 increases during the sleep and the cell size 30 decreases due to loss of intracellular fluid, i.e. water.
B—The patient is awake and drinking and eating, the bio impedance 29 falls while the cell size 30 increases, the cell size increases relatively more than the bio impedance reduction which might indicate water intake with little electrolytes.
C—The patient is hiking, the bio impedance 29 rises, and the cell size 30 falls as the patient loses more fluids than the possible intake.

D—The patient drinks fluid with electrolytes (sports drink) after hiking, both the cell size 30 and the bio impedance 29 have a similar relative change, albeit in opposite directions.

E—The patient maintains hydration level by drinking water, but there is a minute loss of electrolytes

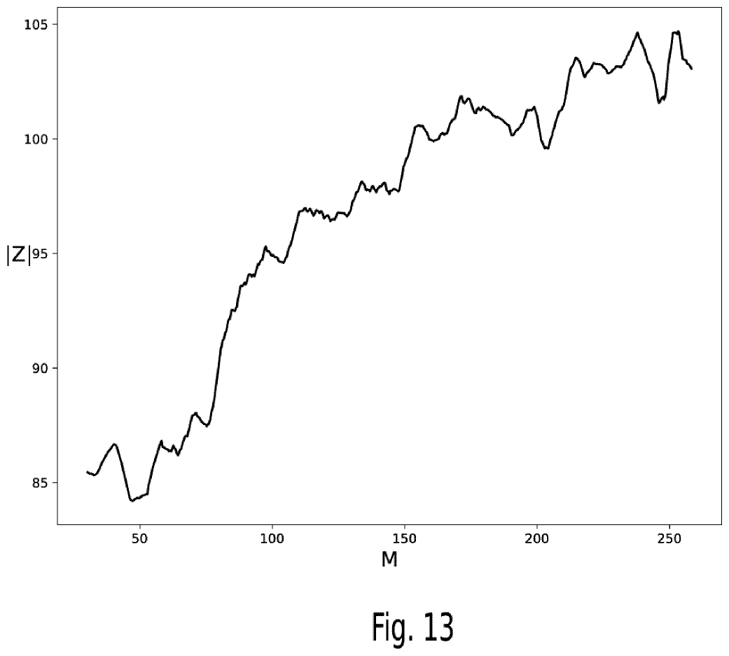

FIG. 13 shows a case where a patient is subjected to an extraction of two litres of liquid from the body and at the same time drinks 0,2 litres of water during a four-hour time period. The result is that the total impedance increases clearly over this time period. This indicates a significant dehydration of the patient. The time course of change in impedance is well correlated with extraction curves (r>0,98). The relative change in impedance can thus be correlated to extracted volume of liquid.

The present invention is a method that can be used by a sensor device to measure hydration in the body, provide an output about the hydration state and provide an alert to any changes in hydration state.

The sensor device is preferably a battery powered, four electrode, light weight, body mountable, flexible patch that makes time resolved electrical resistance and reactance measurements over a wide range of frequencies.

Through these measurements with high time resolution and mathematical modelling intracellular and extracellular volume, in addition to cell volume, and hydration state can be calculated and displayed, and the patch may also provide an alert to changes in hydration state.

The patch is preferably designed to be attached by adhesive to the skin of the patient, at the location of musculus trapezius, approximately at the same level as the heart. This is a good position as it will not interfere with the movement of the patient and is beyond reach of patients who may be inclined to remove foreign objects, such as patients with dementia.

The position is also a central place of the body. Measuring impedance in one muscle group such as musculus trapezius, which does not change much throughout a grown person's lifetime, is considered to be representative of the hydration level of the body as such. Using the trapezius muscle group and placing the patch at the level of the heart will also make the measurements hydrostatically neutral.

Possible medical examples of use can be expressed as:
(1) Any disease, medication or activity that leads to a change in intracellular volume will shift the hydration curve to higher or lower frequencies in the direction of arrow A in FIG. 8.
(2) Any disease, medication or activity that lead to a general change in total body water, will result in a general shift of the measured impedance of the hydration curve in the direction of arrow B in FIG. 8.

(3) Any disease, medication or activity that leads to a change in extra cellular volume only, will shift the measured impedance and shift the low frequency end of the hydration curve.
(4) Any disease, medication or activity that leads to a change in extracellular electrolyte concentration, will shift the measured impedance of the left tail of the hydration curve vertically (see FIG. 8).
(5) Any disease, medication or activity that leads to a general shift in electrolyte concentration, will shift the hydration curve vertically, in the direction of arrow B in FIG. 8.
(6) Any disease, medication or activity that leads to a change in intracellular electrolyte concentration, will shift the hydration curve horizontally in the direction of arrow A in FIG. 8.

Shifts are realized according to the following matrix, making each shift unique and detectable:

| | Hypertone | Isotone | hypotone |
|---|---|---|---|
| Hypervolemia | ¹Na↑ total volume↑ | ⁴Na→ total volume↑ | ⁷Na ↓ total volume↑ |
| Isovolemia | ²Na↑ total volume→ | ⁵Na→ total volume | ⁸Na↓ total volume→ |
| | | → | |
| Hypovolemia | ³Na↑ total volume↓ | ⁶Na→ total volume↓ | ⁹Na↓ total volume↓ |

Volemia is an indication of change in cell volume, where hyper is excess volume, iso is constant volume and hypo is deficient volume, Tone is an indication of electrolyte (mainly Na⁺) concentration, where hyper is excess concentration, iso is constant concentration and hypo is deficient concentration. Isovolema combined with isotone is the normal condition, which the remaining conditions indicates some sort of anomaly.

Future user scenarios are for the observation of disease processes included in the well-known Renin-agiotensin-aldosterone system such as: inflammatory processes (obesity, rheumatic disorders, osteoarthritis, cancer, stroke and heart attack, rhabdomyolysis), hypertension and atherosclerosis, diabetes and pancreatic disease and liver disease. This list is not exhaustive.

1-11. (canceled)

12. A method for determining if a relative difference in cell volume has occurred, with a sensor device having four electrodes, said sensor device being embedded in a single patch that is attachable to the skin of a patient, the method comprising:
performing a first measurement cycle by:
a) applying a current corresponding to a cycle of different predetermined frequencies in time series on an outer pair of the electrodes;
b) measuring a resulting variating potential at each predetermined frequency over an inner pair of the electrodes;
c) calculating a measured impedance from the corresponding measured variating potential at each predetermined frequency;
d) calculating a total impedance absolute value from the corresponding measured impedance at each predetermined frequency; and
e) storing each total impedance absolute value against its corresponding frequency for the first measurement cycle in a memory;
performing one or more subsequent measurement cycles by repeating steps a) to e), wherein each of the one or more subsequent measurement cycles is performed after a predetermined period of time after the first measurement cycle;
reading one or more corresponding frequency value belonging to at least one selected total impedance absolute value of the one or more subsequent measurement cycles;
reading a corresponding frequency value belonging to the same at least one selected total impedance absolute value of the first measurement cycle;
comparing said frequency values of said first and said one or more subsequent measurement cycles, and determining a difference between the frequency values; and
providing a time series of differences in frequency across said first and one or more subsequent measurement cycles;
wherein an increasing frequency difference across said time series indicates a relative increase or decrease of cell volume.

13. The method of claim 12, wherein the total impedance absolute value over the cycle of predetermined frequencies is curve-fitted to an empirical data model; and
wherein parameters α, R_{E} and R_{T} to be used to determine a change of cell volume are extracted from said empirical model, said empirical model being the following approximation:
$$\vert Z ( f ) \vert \approx \frac{\sqrt[γ]{{R}_{E}^{γ} + {( {R}_{T} \cdot α \cdot f )}^{γ}}}{1 + {( α \cdot f )}^{γ}}$$
where |Z(f)| is the total impedance absolute value, γ is a slope parameter derived from the steepness of a magnitude plot determined as an empirical value from a large set of measurements, R_{E} is extra cellular resistance, R_{I} is intracellular resistance, R_{T} is the total resistance, f is the frequency, and α is a frequency dependent factor of the cell volume; and
determining a change in cell volume, the change in cell volume being according to the following relationship:
$$Cell volume \sim \frac{α}{{R}_{E} + {R}_{I}} .$$

14. The method of claim 12, further comprising:
reading respective frequencies belonging to a plurality of selected total impedance absolute values of the first measurement cycle; and
reading respective frequencies belonging to the plurality of selected total impedance absolute values of the one or more subsequent measurement cycle;
determining differences between each frequency value for the same total impedance absolute value for the plurality of selected total impedance absolute values; and
calculating an average frequency difference between the first and the one or more subsequent measurement cycles.

15. The method of claim 12, wherein extra cellular bio impedance and intra cellular bio impedance is determined from a Cole-Cole plot.

16. The method of claim 12, wherein the measured impedance over the cycle of different predetermined frequencies is compared with an upper boundary curve and a lower boundary curve, wherein the boundary curves are determined such that the measured impedance on a higher frequency is allowed to be within A%, typically 90%, to B%, typically 101% ,of a corresponding closest lower frequency and that a series of measured impedance over the cycle of different predetermined frequencies is rejected if any of the measurements fall outside one or both said boundary curves.

17. The method of claim 12, wherein the empirical data model is created using reference measurements using an impedance meter.

18. The method of claim 17, wherein the slope parameter is determined based on said reference measurements.

19. The method of claim 12, wherein a true signal from a signal and noise mix resulting from the measured potential is estimated by passing the signal/noise mix through a Fourier transform for each of said known frequencies, using the following formula:
ŝ= [symbol] (s+n)
where ŝ is an estimate of the signal without noise, s is the pure signal and n is the noise.

20. The method of claim 12, further comprising:
implementing the electrodes and a processor for performing the method steps of any of claims 1 to 8 into a patch to be adhered to the skin of a patient before carrying out the steps of any of claims 1 to 8.

21. The method of claim 12, wherein the calculated estimate of cell volume change and total impedance absolute value change are compared to threshold values and, when any of the cell volume change or the total impedance value change exceeds said threshold values, an alert indicating underhydration or overhydration is sent to a receiver unit.

22. The method of claim 21, wherein a change in either extra cellular bio impedance or intra cellular bio impedance is monitored.

23. A method for determining if a relative difference in cell volume has occurred, with a sensor device having four electrodes, said sensor device being embedded in a single patch that is attachable to the skin of a patient, the method comprising:
performing a first measurement cycle by:
a) applying a current corresponding to a cycle of different predetermined frequencies in time series on an outer pair of the electrodes;
b) measuring a resulting variating potential at each predetermined frequency over an inner pair of the electrodes;
c) calculating a measured impedance from the corresponding measured variating potential at each predetermined frequency;
d) calculating a total impedance absolute value from the corresponding measured impedance at each predetermined frequency; and
e) storing each total impedance absolute value against its corresponding frequency for the first measurement cycle in a memory;
performing one or more subsequent measurement cycles by repeating steps a) to e), wherein each of the one or more subsequent measurement cycles is performed after a predetermined period of time after the first measurement cycle;
reading one or more corresponding frequency value belonging to at least one selected total impedance absolute value of the one or more subsequent measurement cycles;
reading a corresponding frequency value belonging to the same at least one selected total impedance absolute value of the first measurement cycle;
comparing said frequency values of said first and said one or more subsequent measurement cycles, and determining a difference between the frequency values; and
providing a time series of differences in frequency across said first and one or more subsequent measurement cycles;
wherein an increasing frequency difference across said time series indicates a relative increase or decrease of cell volume; and
wherein the total impedance absolute value over the cycle of predetermined frequencies is curve-fitted to an empirical data model; and
wherein parameters α, R_{E} and R_{T} to be used to determine a change of cell volume are extracted from said empirical model, said empirical model being the following approximation:
$$\vert Z ( f ) \vert \approx \frac{\sqrt[γ]{{R}_{E}^{γ} + {( {R}_{T} \cdot α \cdot f )}^{γ}}}{1 + {( α \cdot f )}^{γ}}$$
where |Z(f)| is the total impedance absolute value , γ is a slope parameter derived from the steepness of a magnitude plot determined as an empirical value from a large set of measurements, R_{E} is extra cellular resistance, R_{I} is intracellular resistance, R_{T} is the total resistance, f is the frequency, and α is a frequency dependent factor of the cell volume; and
determining a change in cell volume, the change in cell volume being according to the following relationship:
$$Cell volume \sim \frac{α}{{R}_{E} + {R}_{I}} .$$
